(19) United States
(12) Patent Application Publication
(10) Pub. No.: US 2020/0364887 A1
(43) Pub. Date: Nov. 19, 2020

(54) SYSTEMS AND METHODS USING TEXTURE PARAMETERS TO PREDICT HUMAN INTERPRETATION OF IMAGES

(71) Applicant: University of Houston System, Houston, TX (US)

(72) Inventor: Mini Das, Houston, TX (US)

(73) Assignee: University of Houston System, Houston, TX (US)

(21) Appl. No.: 16/613,921

(22) PCT Filed: May 17, 2018
(86) PCT No.: PCT/US2018/033282

§ 371 Date: Nov. 15, 2019

Related U.S. Application Data
(60) Provisional application No. 62/507,681, filed on May 17, 2017.

(51) Int. Cl. G06T 7/40; G06T 7/00; G06K 9/32; G06N 20/00; G16H 50/20

(52) U.S. Cl. CPC G06T 7/40; G06T 7/0012; G06K 9/3233; G06N 20/00; G06T 2207/10108; G06T 2207/10056; G06T 2207/10088; G06T 2207/10112; G16H 50/20

(57) ABSTRACT

Systems and methods for using texture parameters to predict human interpretation of images to design, modify, or optimize an imaging system or an imaging process. According to embodiments, a method includes receiving image data; calculating one or more texture parameters based on the image data; and predicting performance of a human observer in detecting a signal or object in the image data obtained by the imaging system or processed by the imaging process based on the one or more texture parameters. The method may include designing, optimizing, or modifying parameters of the imaging system or the imaging process based on the predicted performance of a human observer or interpreter in detecting the signal or object in the image data. The method may include assigning weights to two or more texture parameters based on a given detection task, such as detecting explosives in luggage or finding a tumor in tomographic images.

### BACKGROUND

Various modalities (e.g., magnetic resonance imaging (MRI) and computerized tomography (CT)) have been developed to image features within an object, such as imaging bone and soft tissues within the human body or imaging explosives within luggage or packages. These modalities are typically used to generate multiple, cross-sectional slices of the body or item being imaged. A human observer, such as a radiologist or airport security worker, subsequently examines the cross-sectional slices to identify any abnormal structures (e.g., tumors or explosives) that may be present in the body or item.

The performance of human observers in detecting features or structures within images of an object is considered as the gold standard in assessing the quality of images and the best imaging systems. This is critical in areas like medical imaging (perception of a radiologists or a human observer), defense (finding signals in dense or turbulent atmospheres), and security (finding explosives in x-ray images or images from other modalities). However, human observer studies required to predict performance changes due to system design and optimization, selection or modification of software such as filters and algorithms used to smooth or improve images, or any such operations that may be applied to the images may be complicated and time-consuming.

### SUMMARY

In one aspect, this disclosure features a method. The method includes receiving at least one image, calculating one or more texture parameters based on the at least one image, and predicting performance of a human observer, e.g., and average human observer, in detecting a signal or an object in the at least one image obtained by an imaging system or processed by an imaging process based on the one or more texture parameters.

In aspects, the one or more texture parameters are one or more of correlation, homogeneity, energy, entropy, contrast, coarseness, busyness, complexity, short runs emphasis, long runs emphasis, gray level nonuniformity, run length nonuniformity, or run percentage.

In aspects, the method includes selecting a region of interest (ROI) within the at least one image.

In aspects, the at least one image is a simulated image of a patient, a clinical image of a patient, a simulated image acquired by an airport scanner, an actual image acquired by an airport scanner, a tomographic image, a projection image as used in mammography, or a microscopic image.

In aspects, the method includes developing, modifying, or optimizing a machine learning algorithm used in the imaging system, the imaging process, or other image interpretation device based on the predicted detection performance of the human observer; developing, modifying, or optimizing a computer-aided diagnosis (CAD) engine based on the predicted detection performance of the human observer; developing, modifying, or optimizing a search engine based on the predicted detection performance of the human observer; developing, modifying, or optimizing a model for humans based on the predicted detection performance of the human observer; developing, modifying, or optimizing a visual search model based on the predicted detection performance of the human observer; developing, modifying, or optimizing a method or process of forming digital pathological images or microscopic images based on the predicted detection performance of the human observer; or

developing, modifying, or optimizing psychophysical models for search and detection by humans based on the predicted detection performance of the human observer.

In aspects, the method includes assigning weights to two or more texture parameters based on a detection task. The detection task may be detecting explosives in bags or packages, finding a high or low contrast signal in a dense or turbulent atmosphere, finding a signal having high or low spatial frequency, internally inspecting a component, detecting flaws in a product, or finding a mass or calcification in body tissue, a blood vessel, or an organ.

In aspects, the imaging system is an X-ray system, an optical system, an ultrasound system, a three-dimensional ultrasound system, a magnetic resonance imaging (MRI) system, a planar imaging system, a tomographic imaging system, a computed tomography system, a photon-counting computer tomography system, a digital breast tomosynthesis (DBT) system, a photoacoustic or optoacoustic imaging system, e.g., for detecting skin melanoma, a magnetic particle imaging system, a terahertz wave imaging system, a millimeter wave imaging system, an emission computer tomography (ECT) system, a positron emission tomography (PET) system, a single-photon emission computed tomography (SPECT) system, or any combination of two or more of these imaging systems.

In aspects, the imaging process is a process performed by the imaging system.

In aspects, the method includes designing, modifying, or optimizing parameters or structures of the imaging system or the imaging process based on the predicted performance of a human observer to detect a signal or an object in the at least one image.

In aspects, the imaging process is an image filter, an image processing algorithm, an image acquisition method, or an image reconstruction method.

In aspects, the imaging process is a new or modified software method, a planar imaging process, a tomographic imaging process, a partial-angle tomographic imaging process, a scatter-imaging process, image acquisition process, or image reconstruction process. In aspects, the imaging process is a new or modified imaging software application that performs an image acquisition process and/or an image reconstruction process for an existing imaging system.

In another aspect, this disclosure features a system. The system includes an imaging system, a processor, and a memory. The memory has stored thereon instructions which, when executed by the processor, cause the processor to: receive image data from the imaging system, calculate one or more texture parameters based on the received image data, and predict performance of an average human observer in detecting a signal or an object in the image data received from the imaging system or processed by an imaging process based on the one or more texture parameters.

In aspects, the instructions cause the processor to design, modify, or optimize parameters or structures of the imaging system or the imaging process based on the predicted performance of a human observer to detect a signal or an object in the image data.

In aspects, the instructions cause the processor to assign weights to two or more texture parameters based on a detection task.

In aspects, the imaging process is a new or modified software method, image acquisition method, or reconstruction method to be executed on the imaging system, and the instructions cause the processor to: determine whether the predicted performance is greater than a predetermined threshold and, if it is determined that the predicted performance is not greater than the predetermined threshold, modify the software application or parameters or settings of the software application based on the predicted performance.

In yet another aspect, this disclosure features a computer system. The computer system includes a processor and a memory. The memory has stored thereon instructions which, when executed by the processor, cause the processor to: receive image data from an imaging system, calculate one or more texture parameters based on the received image data, and predict performance of an average human observer in detecting a signal or an object in the image data received from the imaging system or processed by an imaging process based on the one or more texture parameters.

In aspects, the instructions cause the processor to design, modify, or optimize parameters or structures of the imaging system or the imaging process based on the predicted performance of a human observer to detect a signal or an object in the image data.

In aspects, the instructions cause the processor to assign weights to two or more texture parameters based on a detection task.

In aspects, the imaging process is an image filter, an image processing algorithm, an image acquisition method, or an image reconstruction method.

### BRIEF DESCRIPTION OF THE DRAWINGS

For a detailed description of various examples, reference will now be made to the accompanying drawings, in which:

FIG. 1 depicts an illustrative computer system for performing the techniques described herein, in accordance with various embodiments;

FIG. 2 depicts a flow diagram illustrating a method in accordance with some embodiments;

FIG. 3 depicts a table listing texture parameters that may be calculated by the computer system of FIG. 1 using the methods of FIGS. 2, 4, and 5, in accordance with various embodiments;

FIG. 4 depicts a flow diagram illustrating a method in accordance with other embodiments; and

FIG. 5 depicts a flow diagram illustrating a method in accordance with yet other embodiments.

### DETAILED DESCRIPTION

New imaging modalities and processes are often developed for a variety of purposes, including the purposes described above. The quality and usefulness of each such modality may be ascertained to some extent based on human interpreters' ability to identify abnormalities in the images with a high degree of sensitivity and specificity. It is impractical, however, to have human interpreters read images for this purpose, as such images may number in the thousands or hundreds of thousands. As a result, models, such as mathematical models, have been developed to predict the manner in which human interpreters would read the images. The outputs of such models are used to determine sensitivity and specificity parameters.

These models usually require prior information of system design (to generate images via simulations) or need a large amount of collected data to model human detection. These models often try to emulate localization and detection of signal via methods like Hotelling Observers or visual search observers. These models also need insertion of signals (either as simulated images or as hybrid images). Consequently, these models tend to be unduly complex, thus increasing costs and logistical burdens. Also, it is often impractical to test new imaging systems based on clinical data or data from airport scanners. Thus, many new clinical systems use simulated images to develop predictions. Further, once clinical systems are installed, there is currently no effective way to test whether changes to the processing of acquired clinical images by the clinical systems or changes to the processing parameters of the clinical systems can improve the detection of signals or objects in the acquired clinical images.

The systems and methods of this disclosure predict the manner in which human interpreters or observers would read images, but they mitigate the aforementioned disadvantages associated with the model-based approach. More specifically, the systems and methods generally entail the calculation and evaluation of certain texture parameters using image data produced by the imaging modality being tested or designed. These texture parameters, including first-order or second-order texture parameters such as contrast, gray level nonuniformity, complexity, and entropy, strongly correlate with human interpretation of images and thus can serve as simpler, more efficient, automatically-calculated (e.g., using executable code stored on non-transitory computer-readable media) proxies for human interpretation of images. They can thus be used to evaluate emerging imaging modalities and to read images to identify pathological and non-pathological abnormalities. The systems and methods of this disclosure present a superior alternative to the aforementioned model-based approaches at least because they provide the technical advantage of increased efficiency.

This disclosure provides simple to implement methods that can be used in pre-design of an imaging system or an imaging process, such as image acquisition or image reconstruction. The methods can also be used in the post-installation of an imaging process, e.g., image acquisition software and/or image reconstruction software. The pre-designed imaging system or process or the post-installation process can be tested through simple calculations that may involve weighting texture parameters. In embodiments, different weights may be applied to the texture parameters depending on the spatial frequency of a signal being detected. For example, detection of low-contrast masses (e.g., cancers) or microcalcification clusters in breast images may require different weightings for the selected texture features. The systems and methods of this disclosure can take simulated images or clinical images of a patient and estimate a series of texture parameters (e.g., grey level nonuniformity, complexity, and contrast), assign weights to them based on the detection task to be performed, and make predictions about which imaging system or software would improve signal or object detection.

Although this disclosure often describes the systems and methods of this disclosure in the context of medical imaging, the systems and methods of this disclosure may be adapted for application in any suitable context (e.g., airport security scanners, signal detection in the atmosphere, general search engine development, or computer-aided diagnosis (CAD) engine development). Also, as used in this disclosure, the term “imaging process” includes “image processing”.

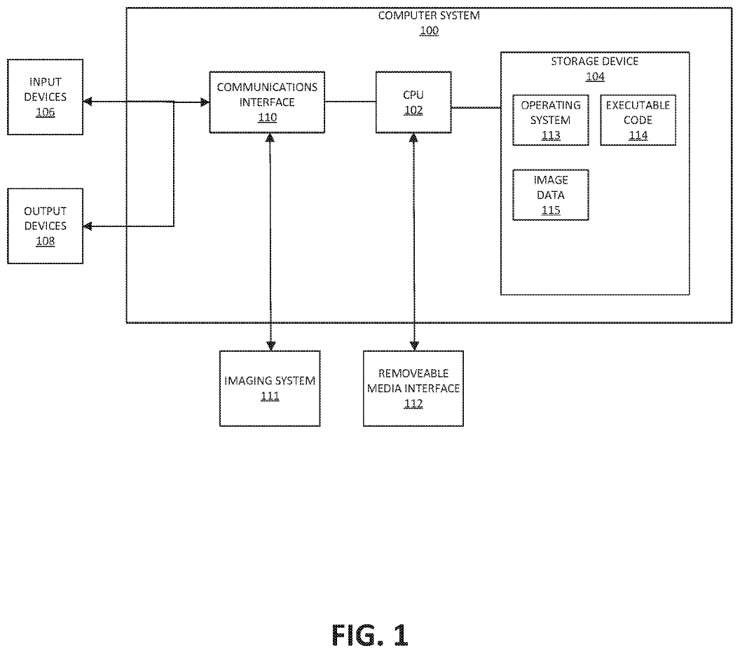

FIG. 1 depicts an illustrative computer system 100 for performing the techniques or methods described herein, in accordance with various embodiments. The computer system 100 includes a central processing unit (CPU) 102 that couples to storage device 104, one or more input devices 106 (e.g., keyboard, mouse, touchscreen, microphone), and one or more output devices 108 (e.g., display, speaker). The CPU 102 may also couple to a communications interface 110 that facilitates communications with one or more other electronic devices via a network, such as the Internet or a local area network. The CPU 102 may couple to an imaging system 111 via the communications interface 110 to receive images obtained by the imaging system 111 and to store the received images in the storage device 104. The imaging system 111 may be an X-ray system, an optical system, an ultrasound system, a three-dimensional ultrasound system, a magnetic resonance imaging (MRI) system, a planar imaging system, a tomographic imaging system, a computed tomography system, a photon counting computer tomography system, a digital breast tomosynthesis (DBT) system, a photoacoustic or optoacoustic imaging system, a magnetic particle imaging system, a terahertz wave imaging system, a millimeter wave imaging system, an emission computer tomography (ECT) system, a positron emission tomography (PET) system, a single-photon emission computed tomography (SPECT) system, or any combination of two or more of these imaging systems.

The CPU 102 may further couple to a removable media interface 112, such as a thumb drive receptacle (e.g., a universal serial bus (USB) connector). The computer system 100 may contain or couple to various other components that facilitate performance of the operations described herein. Storage device 104 may be a non-transitory computer-readable storage medium that stores operating system code 113, executable code 114, and image data 115, e.g., image data acquired by the imaging system 111 and stored in the storage device 104 by the CPU 102. The executable code 114, when executed by the CPU 102, causes the processor 102 to perform some or all of the operations described herein.

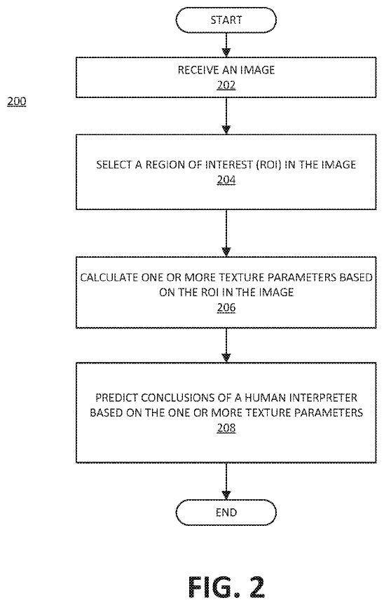

FIG. 2 depicts a flow diagram illustrating a technique or method 200 in accordance with some embodiments. The CPU 102 may perform some or all of the method 200 as a result of executing the executable code 114. The method 200 includes receiving an image (block 202). The image may have been generated using any suitable imaging modality, including novel imaging modalities being tested. The image may include a simulated image of a patient, a clinical image of a patient, a simulated image acquired by an airport scanner, an actual image acquired by an airport scanner, a tomographic image, a projection image as used in mammography, or a microscopic image. The CPU 102 may acquire the image from another electronic device via the network interface 112, for example. Alternatively, the CPU 102 may execute instructions to acquire the image from storage device 104 or from a removable storage coupled to the removable media interface 112. In at least some embodiments, the acquired image includes a grayscale image, although other types of images are contemplated, e.g., an RGB image.

The method 200 then includes the CPU 102 selecting a region of interest (ROI) (block 204). The ROI may be where the observer would be searching for a signal or an object. The ROI may be selected by the CPU 102 automatically or by a user instructing the CPU 102 to select the ROI using an input device 106. The ROI may be of any suitable size. For example, the ROI may be 30 pixels×30 pixels, or it may be 100 pixels×100 pixels. In some cases, multiple ROIs may be selected, and the remainder of the method 200 may be performed for each of the multiple ROIs. The method 200 then includes the CPU 102 calculating one or more texture parameters based on image data from the ROI (block 206).

The raw intensity values present in the ROI may be quantized into any suitable number of possible gray level values as part of block 206. In some embodiments, the raw intensity values are quantized into 256 separate possible gray level values, each of which may be represented by, e.g., an 8-bit digital code. In other embodiments, the number of possible gray level values may depend on the task to be performed. For example, a large number of gray level values may be needed to detect a particular object or type of object in an image.

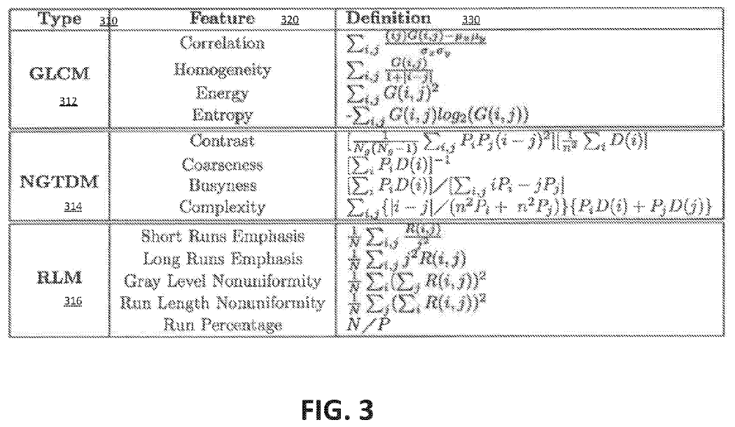

The quantization thresholds may be user-defined or may be pre-programmed into the executable code 114. Also as part of block 206, the CPU 102 may use the quantized values to calculate any suitable number of texture parameters. FIG. 3 depicts a table listing texture parameters (identified under the “Feature” column 320) that may be calculated by the computer system 100 of FIG. 1 using the method 200 of FIG. 2, in accordance with various embodiments. Each of the texture parameters may be calculated based on a spatial co-occurrence matrix, which is also depicted in FIG. 3 under the “Definition” column 330.

Referring to FIG. 3, three different types of spatial co-occurrence matrices are listed under the “Type” column 310: the gray level co-occurrence matrix (GLCM) 312, the neighborhood gray tone difference matrix (NGTDM) 314, and the run length matrix (RLM) 316. The GLCM 312 details the spatial relationship between gray levels across an image, and it requires a direction in which to compare pixels (e.g., 0 degrees, 45 degrees, 90 degrees, 135 degrees). FIG. 3 describes the texture parameters that are associated with GLCM 312, including correlation, homogeneity, energy, and entropy, as well as the mathematical expressions to calculate each such texture parameter. For such parameters, G (i, j) represents the normalized GLCM entry at gray levels i and j. This represents the probability of co-occurrence of gray levels i and j across the entire region of interest (ROI) in a specified direction (e.g., 45 degrees). Any sum over i, j signifies a sum over all permutations of i and j. The terms μₓ and μ_{y} in the expression for correlation represent the mean value of the rows and columns, respectively, for G(i, j). Similarly, σₓ and σ_{y} represent the standard deviations of these rows and columns.

The NGTDM 314 details information about the “neighborhood” of gray levels that surround a certain intensity value. For example, a high NGTDM value generally indicates that a certain gray level is significantly different from its surrounding neighborhood. Texture parameters that may be calculated in association with the NGTDM 314 include contrast, coarseness, busyness, and complexity, and FIG. 3 describes the mathematical expressions to calculate each such parameter. The NGTDM S(i) is a vector of dimension N_{g}×1 (where N_{g} is the number of gray levels) that details the degree of difference between pixels of a certain gray level i (with probability of occurrence Pᵢ) and its surrounding neighborhood of n² pixels. Accordingly, a relatively high value of S(i) indicates that, across an entire ROI, the gray level i differs significantly from its surrounding pixels at one or more locations.

The RLM 316 describes the frequency of runs of identical pixel values for a given length. It requires a specific direction, as is true of the GLCM 312. Texture parameters that may be calculated with respect to the RLM 316 include short runs emphasis, long runs emphasis, gray level nonuniformity, run length nonuniformity, and run percentage. With regard to the RLM 316, for a given gray level i, R(i,j) is the number of consecutive pixel runs of length j in a specified direction across an ROI. In addition, N denotes the sum of all RLM elements, and P is the total number of pixels in the image being analyzed.

Referring again to FIG. 2, although the CPU 102 may calculate any of the aforementioned texture parameters for a particular ROI in a particular image, in some embodiments, the CPU 102 only calculates the texture parameters (or a subset) that are shown to have the greatest correlation (whether positive or negative) to human interpreter performance—e.g., the contrast, gray level nonuniformity, complexity, and entropy texture parameters (in some embodiments, a fifth parameter—homogeneity—may be used). The processor 102 may use the values obtained for the texture parameters to predict the conclusions that a human interpreter, such as a radiologist, would have drawn when visually inspecting the same image and, in particular, the same ROI (block 208). For example, the executable code 114 may be programmed with texture parameter thresholds such that when a particular texture parameter exceeds a relevant threshold, the processor 102 makes a different prediction than it would have had that particular texture parameter not exceeded that particular threshold.

In some embodiments, the processor 102 makes predictions based on the success or failure of multiple texture parameters crossing associated thresholds. For instance, if the CPU 102 determines that both the contrast and gray level nonuniformity values exceed certain thresholds, it may predict that the ROI contains an abnormality warranting further attention. The method 200 may be adjusted as desired, including by adding, deleting, modifying, or rearranging one or more steps.

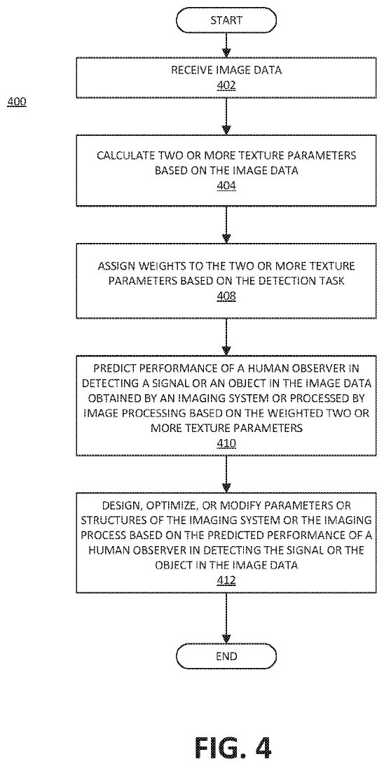

FIG. 4 depicts a flow diagram illustrating a method 400 in accordance with other embodiments. The CPU 102 may perform some or all of the method 400 as a result of executing the executable code 114. The method 400 includes receiving image data (block 402). The method 400 then includes calculating two or more texture parameters based on the image data (block 404). The two or more texture parameters may include two or more texture parameters listed in FIG. 3 and/or other suitable texture parameters. The method 400 next includes assigning weights to the one or more texture parameters based on the detection task (block 406). For example, a weight applied to the contrast texture parameter for tumor detection may be smaller than a weight applied to the contrast texture parameter for detecting explosives. The detection tasks may include detecting an explosive within a suitcase or a package and detecting a tumor, abnormal mass, or calcification within an anatomical feature of a patient.

The method 400 next includes predicting performance of a human observer in detecting a signal or an object in the image data obtained by an imaging system or processed by image processing based on the weighted two or more texture parameters (block 410). Then, the parameters of the imaging system or the imaging process are optimized or modified based on the predicted performance of a human observer in detecting the signal or the object in the image data (block 412).

The parameters of the imaging system that are optimized or modified based on the predicted performance may include the type of modality (e.g., X-ray, optical, ultrasound, MRI, or a combination of two or more of these modalities) used, parameters for operating the selected type of modality, the angular span, the number of projections along the angular span, the imaging time, the total imaging time, or the number of photon counts required to achieve best results. The parameters of the imaging process may include filter coefficients, the type of acquisition method, the type of reconstruction method (e.g., filtered backprojection (FBP), expectation maximization (EM), or total variation (TV)-minimization), or the type of filter (e.g., Butterworth, Chebyshev, Bessel, Elliptic, smoothing, or edge preserving). Any one or more of these parameters may be optimized or adjusted based on predicted performance of a human observer in detecting a signal or an object in image data.

For tomographic images, the CPU 102 may determine whether tomographic slices should be binned or slabbed—to reduce the number of slices presented to the observer or radiologist—based on the predicted performance of a human observer to detect a signal or object in the binned or slabbed slice. Binning involves taking a matrix of pixels and combining the pixels to create one larger pixel. Binning may be performed in the hardware, e.g., the X-ray detector. And slabbing involves combining two or more slices to generate a new thicker slice.

In some embodiments, the CPU 102 may determine optimal parameter values and/or methods to use for the binning or slabbing based on the predicted performance of a human observer to detect a signal or object in the binned or slabbed slice. For example, slabbing may be performed by using the maximum pixel value or the arithmetic mean of the pixels of interest. The relevant texture features, which may be weighted based on task of detecting microcalcifications, may indicate that using the maximum pixel value provides better performance in detecting microcalcifications than using the arithmetic mean. On the other hand, the relevant texture features, which may be weighted based on the task of detecting low-density objects, may indicate that using the arithmetic mean provides better performance in detecting low-density objects with a lower noise than using the maximum pixel value. Thus, in embodiments of this disclosure, various parameters, settings, or structures of an imaging system or process can be selected and/or modified to optimize detection performance by a human observer.

In embodiments, constraints may be placed on the range of values over which the parameters of the imaging system or process can be adjusted or modified. In some embodiments, characteristics of the patient, e.g., patient size, may place constraints on the parameters of the imaging system or process. For example, operational parameters such as current and voltage used in X-ray imaging systems may vary for heavy patients, slim patients, adult patients, or pediatric patients. In particular, for pediatric patients, it is critical to minimize or reduce imaging time and dose. Thus, for example, the current and voltage used in performing X-ray imaging of a pediatric patient may be limited to low current and voltage ranges and short imaging times.

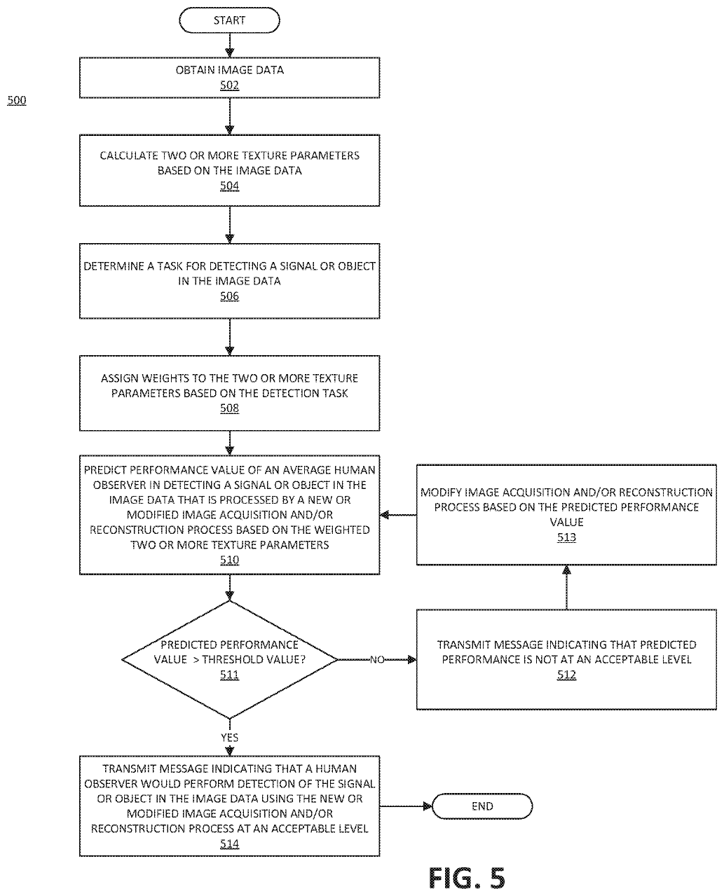

FIG. 5 depicts a flow diagram illustrating a method 500 in accordance with yet other embodiments. The CPU 102 shown in FIG. 1 may perform some or all of the method 500 as a result of executing the executable code 114. The method 500 includes obtaining image data (block 402). The image data, e.g., the image data 115 shown in FIG. 1, may be obtained or retrieved by the CPU 102 from the storage device 104. The method 500 then includes calculating two or more texture parameters based on the image data (block 504). The two or more texture parameters may include two or more texture parameters listed in FIG. 3 and/or other suitable texture parameters. The method 500 next includes determining a task for detecting a signal or object in the image data (block 506). The detection tasks may include detecting an explosive within a suitcase or package and detecting a tumor, abnormal mass, or calcification within an anatomical feature of a patient. The method 500 next includes assigning weights to the one or more texture parameters based on the detection task (block 508).

The method 500 next includes predicting performance of a human observer in detecting a signal or object in the image data that is processed by an image acquisition and/or reconstruction process based on the weighted two or more texture parameters (block 510). Next, the method 500 includes determining whether the predicted performance value is greater than a predetermined threshold value (block 511). The predetermined threshold may be set based on a range of performance values that are based on an acceptable level of accuracy in detecting the signal or object in the image data. If it is determined that the predicted performance value is not greater than a predetermined threshold value, the method 500 includes transmitting a message indicating that the predicted detection performance is not at an acceptable level (block 512), modifying the image acquisition and/or reconstruction process based on the predicted performance value (block 513), and re-predicting the performance value using the modified image acquisition and/or reconstruction process (block 510).

If, on the other hand, it is determined that the predicted performance value is greater than a predetermine threshold, the method 500 includes transmitting a message indicating that a human observer would perform detection of the signal or object in the image data using the new or modified image acquisition and/or reconstruction process at an acceptable level, e.g., an acceptable level of accuracy (block 514).

In some embodiments, the computer system 100 of FIG. 1 may include executable code 114, which, when executed by the CPU 102, causes the CPU 102 to: determine whether the predicted performance value is greater than a predetermined threshold value, and, if the predicted performance value is not greater than the predetermined threshold value, prevent use or installation of a new or modified software application that performs the image acquisition and/or reconstruction process.

Additionally or alternatively, if the predicted performance value is not greater than the predetermined threshold value, the executable code 114 may cause the CPU 102 to generate and transmit a message, e.g., a warning message, to one of the output devices 108 shown in FIG. 1 indicating that the new or modified software application for performing an image acquisition and/or reconstruction process may make it difficult or challenging for a human observer to detect a signal or object in images processed using the new or modified software application that performs an image acquisition and/or reconstruction process, or image reconstruction process.

Additionally or alternatively, if the predicted performance value is not greater than the predetermined threshold value, the executable code 114 may cause the CPU 102 to generate and transmit a message to one of the output devices 108 suggesting possible changes to the parameters of the software application that performs an image acquisition and/or reconstruction process, or suggesting other new software applications that performs the same or other image acquisition processes and/or image reconstruction processes.

In embodiments, the predicted performance of a human observer in detecting a signal or object in an image may be used to develop, modify, or optimize a variety of systems, methods, processes, or algorithms for a variety of applications. The systems, methods, processes, or algorithms that may be developed, modified, or optimized based on predicted performance of a human observer in detecting a signal or object in images include: a machine learning algorithm used in an imaging system, an imaging process, or other imaging interpretation device; a computer-aided diagnosis (CAD) engine, a search engine, a model for human observers; a visual search model; a method or process of forming digital pathological images or microscopic images; and psychophysical models for search and detection by humans.

The above description is meant to be illustrative of the principles and various examples of the present disclosure. Numerous variations and modifications will become apparent to those skilled in the art once the above disclosure is fully appreciated. It is intended that the following claims be interpreted to embrace all such variations and modifications.

What is claimed is:

1. A method, comprising:
receiving at least one image;
calculating one or more texture parameters based on the at least one image; and
predicting performance of a human observer in detecting a signal or an object in the at least one image obtained by an imaging system or processed by an imaging process based on the one or more texture parameters.

2. The method of claim 1, wherein the one or more texture parameters are one or more of correlation, homogeneity, energy, entropy, contrast, coarseness, busyness, complexity, short runs emphasis, long runs emphasis, gray level nonuniformity, run length nonuniformity, or run percentage.

3. The method of claim 1, further comprising selecting a region of interest (ROI) within the at least one image.

4. The method of claim 1, wherein the at least one image is a simulated image of a patient, a clinical image of a patient, a simulated image acquired by an airport scanner, an actual image acquired by an airport scanner, a tomographic image, a projection image as used in mammography, or a microscopic image.

5. The method of claim 1, further comprising:
developing, modifying, or optimizing a machine learning algorithm used in the imaging system, the imaging process, or other image interpretation device based on the predicted detection performance of the human observer;
developing, modifying, or optimizing a computer-aided diagnosis (CAD) engine based on the predicted detection performance of the human observer;
developing, modifying, or optimizing a search engine based on the predicted detection performance of the human observer;
developing, modifying, or optimizing a model for human observers based on the predicted detection performance of the human observer;
developing, modifying, or optimizing a visual search model based on the predicted detection performance of the human observer;
developing, modifying, or optimizing a method or process of forming digital pathological images or microscopic images based on the predicted detection performance of the human observer; or
developing, modifying, or optimizing psychophysical models for search and detection by humans based on the predicted detection performance of the human observer.

6. The method of claim 5, further comprising assigning a weight to each of two or more texture parameters based on a detection task.

7. The method of claim 6, wherein the detection task is detecting explosives in bags or packages, finding a high or low contrast signal in a dense or turbulent atmosphere, finding a signal having high or low spatial frequency, internally inspecting a component, detecting flaws in a product, or finding a mass or calcification in body tissue, a blood vessel, or an organ.

8. The method of claim 1, wherein the imaging system is an X-ray system, an optical system, an ultrasound system, a three-dimensional ultrasound system, a magnetic resonance imaging (MRI) system, a planar imaging system, a tomographic imaging system, a computed tomography system, a photon-counting computer tomography system, a digital breast tomosynthesis (DBT) system, a photoacoustic or optoacoustic imaging system, a magnetic particle imaging system, a terahertz wave imaging system, a millimeter wave imaging system, an emission computer tomography (ECT) system, a positron emission tomography (PET) system, a single-photon emission computed tomography (SPECT) system, or any combination of two or more of these imaging systems.

9. The method of claim 8, wherein the imaging process is a process performed by the imaging system.

10. The method of claim 1, further comprising designing, modifying, or optimizing parameters or structures of the imaging system or the imaging process based on the predicted performance of a human observer to detect a signal or an object in the at least one image.

11. The method of claim 1, wherein the imaging process is an image filter, an image processing algorithm, a planar imaging process, a tomographic imaging process, a partial-angle tomographic imaging process, a scatter-imaging process, an image acquisition process, or an image reconstruction process.

12. The method of claim 1, wherein the imaging process is a new or modified imaging software application that performs an image acquisition process and/or an image reconstruction process for an existing imaging system.

13. A system, comprising:
an imaging system;
a processor; and
a memory having stored thereon instructions which, when executed by the processor, cause the processor to:
receive image data from the imaging system;
calculate one or more texture parameters based on the received image data; and
predict performance of an average human observer in detecting a signal or an object in the image data received from the imaging system or processed by an imaging process based on the one or more texture parameters.

14. The system of claim 13, wherein the instructions further cause the processor to design, modify, or optimize parameters or structures of the imaging system or the imaging process based on the predicted performance of a human observer to detect a signal or an object in the image data.

15. The system of claim 13, wherein the instructions further cause the processor to assign weights to two or more texture parameters based on a detection task.

16. The system of claim 13, wherein the imaging process is a new or modified software application that performs an image acquisition process or a reconstruction process to be executed on the imaging system, and
wherein the instructions further cause the processor to:
determine whether the predicted performance is greater than a predetermined threshold; and
if it is determined that the predicted performance is not greater than the predetermined threshold, modify the software application or parameters or settings of the software application based on the predicted performance.

17. A computer system, comprising:
a processor; and
a memory having stored thereon instructions which, when executed by the processor, cause the processor to:
receive image data from an imaging system;
calculate one or more texture parameters based on the received image data; and
predict performance of an average human observer in detecting a signal or an object in the image data received from the imaging system or processed by an imaging process based on the one or more texture parameters.

18. The computer system of claim 17, wherein the instructions further cause the processor to design, modify, or optimize a parameter or structure of the imaging system or the imaging process based on the predicted performance of a human observer to detect a signal or an object in the image data.

19. The computer system of claim 18, wherein the structure is defined by image acquisition geometry.

20. The computer system of claim 17, wherein the instructions further cause the processor to assign weights to two or more texture parameters based on a detection task.

21. The computer system of claim 17, wherein the imaging process is an image filter, an image processing algorithm, a planar imaging process, a tomographic imaging process, or a partial-angle tomographic imaging process, a scatter-imaging process, an image acquisition method, or an image reconstruction method.
